(19) United States
(12) Patent Application Publication
(10) Pub. No.: US 2002/0115987 A1
(43) Pub. Date: Aug. 22, 2002

(54) Needle cannula removal by extraction

(75) Inventors: Roger L. Hildwein, Woodinville, WA (US); William Robert Van Lew, JR., Renton, WA (US); Glenn D. Austin, Seattle, WA (US)

Correspondence Address: BANNER & WITCOFF, 1001 G STREET N W, SUITE 1100, WASHINGTON, DC, 20001, US

(73) Assignee: PATH, Seattle, WA (US)

(21) Appl. No.: 10/073,172

(22) Filed: Feb. 13, 2002

Related U.S. Application Data
(60) Provisional application No. 60/268,883, filed on Feb. 16, 2001.
(60) Provisional application No. 60/294,004, filed on May 30, 2001.

(51) Int. Cl. A61B 17/00

(52) U.S. Cl. 606/1; 128/917

(57) ABSTRACT

In a needle cannula extraction device, an engagement stroke moves one engagement member toward another engagement member so as to first engage a needle cannula inserted therebetween. Movement of at least one of the engagement members during a subsequent extraction stroke maintains a generally constant spacing between the engagement members that generally corresponds to a thickness of the needle. The extraction stroke pulls the needle cannula from its hub or other attachment structure, which is held in position. In first and second embodiments, the two engagement members first come together to grip the cannula, and then move together as a unit (with the engaged needle cannula) away from the retained attachment structure. In third and fourth embodiments, a first engagement member is pivotable toward a second engagement member, provided in the form of a backing member, and then away from the retained attachment structure. Either one of the engagement members may be mounted for translational movement away from the other, and spring biased, in such a manner as to maintain the generally constant spacing between the engagement members during the extraction stroke. By maintaining a generally constant spacing between the engagement members which corresponds to the thickness of the needle cannulas to be engaged, needle cannulas within a range of thicknesses can be reliably gripped and extracted.

### CROSS-REFERENCE TO RELATED APPLICATIONS

This application claims the benefit of co-pending U.S. Provisional Applications Serial Nos. 60/268,883 (filed Feb. 16, 2001) and 60/294,004 (filed May 30, 2001).

### FEDERALLY SPONSORED RESEARCH AND DEVELOPMENT

This invention was made in part with government support under Cooperative Agreement No. HRN-A-00-96-90007 awarded by the Agency for International Development. The U.S. Government has certain rights in this invention.

### FIELD OF THE INVENTION

This invention relates to devices for extracting contaminated needles from a syringe or other component, and for rendering a contaminated needle unusable.

### BACKGROUND OF THE INVENTION

Needlestick (sharps) injury and the resulting disease infection are a serious health risk worldwide. Studies have shown that bloodborne diseases such as hepatitis B and C (hep B, C) and human immunodeficiency virus (HIV) are transmitted through needlestick injuries. It has been estimated that 12,000 health care workers are infected with hep B and C through needlesticks each year in the United States; 200 to 300 of these victims may die from bloodborne diseases (source: Stark, Pete. “Health Care Worker Protection Act: Statistics and Talking Points.” http://www.house.gov/stark/documents/needlesticktp.html (May 26, 2000)). In developing countries, the problem is even more acute. Disposal methods and equipment are inadequate (or simply do not exist) in many settings, and used needles and syringes are often found mixed with non-hazardous waste in open piles or pits where children, animals, and trash pickers are exposed to injury.

New policies in both developed and developing countries increase the need for rendering needles harmless at or after the time-of-use. The U.S. Occupational Safety and Health Administration (OSHA) prohibits health workers from recapping needles after use, instead requiring that the needles be placed directly in sharps-disposal boxes. If boxes are unavailable, or syringes and needles are mixed accidentally with other waste, the uncapped, contaminated needles represent a significant risk of needlestick injury and infection. In developing countries, new policies requiring the use of auto-disable syringes to prevent reuse have been instituted. Because former practices often included the reuse of syringes and needles, this policy increases the number of contaminated sharps found in the waste stream. Increased volumes burden the already inadequate waste-disposal systems.

At present, there are at least three basic methods of attaching a needle cannula to a syringe tip. A first method of attachment uses a “Luer-slip” that relies on a friction fit between a tapered female hub and a tapered male syringe tip to maintain a needle/syringe connection. A second Luer-type connection is commonly called a “Luer lock.” In a Luer-lock, flanges of a removal hub are threaded into an internally threaded end of a syringe tip. Both of the “Luer” methods utilize a molded plastic hub to which a needle cannula is integrally molded or otherwise attached, e.g., with adhesive. A third method of attachment employs a hubless design in which a needle cannula is affixed directly to the syringe tip, e.g., by integral molding or adhesive.

To prevent needlestick injury and reuse of contaminated needles, several approaches are known for detaching a used needle cannula from its syringe, or otherwise rendering it less harmful prior to disposal. A needle “burner” is used to incinerate needle cannulas. Cutter devices which cut the needle cannula at the syringe tip, or cut the syringe barrel, are also known. While needlestick injury and/or contaminated needle reuse may be reduced by these methods, these approaches have several shortcomings. For example, needle burners generally operate on electricity, which may not be available in all areas (particularly in developing countries). Needle cutting devices are generally designed for a specific group of needle sizes and/or types. To the extent these are relied upon, medical facilities, clinics and the like may need to keep several types of cutting devices on hand. In addition, cutting the syringe barrel or needle cannula may cause splatter, spray, and aerosolization of the fluid contained inside the needle. Finally, both needle burners and cutting devices typically leave a portion of the needle cannula attached to the syringe tip, thus presenting a continuing health risk.

Needle “stripper” devices represent another class of devices used to lower the health risks of used syringes employing a removable needle cannula/hub combination. A needle/hub combination which is separable from its syringe for disposal, such as by a Luer-slip or Luer-lock, decreases the volume of waste generated by greatly decreasing air spaces and increasing packing density compared to needles and syringes discarded in the same container. Also, the physical removal of the hub/needle combination allows the syringe to be disposed of as infectious waste only, and only the hub and attached needle cannula will require the more expensive handling associated with infectious-sharps waste disposal. One type of needle stripper separates (or “pops”) a Luer-slip hub from a mating syringe tip. Another type of needle stripper twists or unwinds a flanged Luer-lock hub from an internally threaded syringe tip. These types of needle strippers provide benefits over manual removal of needles with Luer-type hubs, such as increased efficiency and reduced risk of needle sticks—by avoiding the need for hand contact with the needle/hub during the removal operation.

While in many ways representing an improvement over burning and cutting devices, needle strippers for Luer-slip and Luer-lock hubs do not disable the used needle/hub assembly. As a result, there is a remaining health risk that the needle/hub assembly may be improperly re-used on another syringe barrel. A further shortcoming of needle stripper devices is that they are designed for use in removing a particular type of hub from a syringe, e.g., Luer-slip or Luer-lock, and cannot be used to remove a needle cannula which is directly attached to a syringe tip.

The present inventors recognized that a needle extraction device that would extract a needle cannula from its associated hub or syringe tip could provide certain advantages over the above-described needle “stripper” devices. For example, a needle extraction device could advantageously function independently of the type of needle attachment, e.g., Luer-slip, Luer-lock, and direct needle/syringe attachment. As the needle hub could be retained with the syringe and only the needle cannula need be disposed as infectious sharps waste, such a needle extracting device could achieve a further reduction in the volume of infectious waste. A needle extraction would also positively disable the needle/hub combination, thus preventing reuse of contaminated needles.

Atsumi U.S. Pat. No. 5,588,966 (see FIG. 11 thereof) discloses a device for removing a needle from its associated hub. Two rotatable members are arranged to engage a needle cannula during simultaneous downward rotation of the members. Downward rotation of the rotatable members is actuated by pressing the syringe downwardly thereagainst. Theoretically, the needle is pulled from its hub as the rotatable members grip the needle and continue to rotate downwardly. Operational difficulties with such a device are evident, however. In particular, due to their pivoting configuration, the spacing between the gripping jaws of the rotatable members initially decreases, then increases, during an operational stroke. It thus appears that without very closely maintained dimensions and tolerances (of the device elements as well as the needles being removed) a device as disclosed would be prone to jamming of the gripping jaws against the needle, generation of insufficient gripping force, and/or premature needle release. In addition, it is apparent that such a device would be quite limited in the amount of pulling force that could be generated, such that removal of large needles would be difficult if not impossible.

### SUMMARY OF THE INVENTION

The present invention provides devices for removing a needle cannula from a hub, a syringe, or other attachment structure. Two sequential stages of motion, an engagement stroke and a gripping stroke, cause a needle cannula to first be engaged between two engagement members, and then extracted from its attachment structure by at least one of the engagement members firmly gripping and pulling the needle cannula away from the attachment structure. Provision is made for maintaining a generally constant spacing between the engagement members, which is determined by a thickness of the needle cannula, throughout the extraction stroke. The inventive devices are thus capable of reliably removing needle cannulas of different thicknesses.

According to the present invention, a device for removing a needle cannula from an attachment structure has a body defining a passageway allowing passage of a needle cannula therethrough while an attachment structure associated with the cannula is restrained in a first position relative to the body. First and second engagement members are provided. At least one of the engagement members is movable, in an engagement stroke, to cause engagement between the engagement members of a needle cannula extending along the passageway. At least one of the engagement members is movable, in an extraction stroke following the engaging stroke, away from the first position while firmly gripping the needle cannula engaged between the engagement members. The movement during the extraction stroke is such as to maintain a generally constant spacing between the engagement members during the extraction stroke. The generally constant spacing varies in relation to a thickness of the engaged needle cannula, such that engaged needle cannula of different thicknesses may be firmly gripped throughout the extraction stroke.

In first and second embodiments of the invention, a needle cannula is inserted into an orifice and between a pair of needle gripping surfaces, while the needle hub or syringe tip is abutted against a surface surrounding the orifice. A first rotation of an operation handle pivots a proximal linkage about a proximal pivot axis such that a gripping surface of the proximal linkage advances towards a gripping surface of a distal linkage. In this manner, the needle cannula is securely gripped between the two needle gripping surfaces. With the needle cannula firmly gripped, a continued rotation of the handle in the same direction rotates the distal and proximal linkages together (as a unit) about a distal pivot axis. During this second portion of handle rotation (an extraction stroke), the needle cannula is pulled downwardly while the hub (or syringe tip) is retained outside (above) the orifice. The needle cannula remains locked securely between the gripping surfaces throughout the entire extraction stroke, whereby the needle cannula is reliably extracted from its associated hub or syringe tip. The needle cannula may then be released to fall harmlessly into an attached container. Biasing members then return the distal and proximal linkages, and the handle, to their original starting positions, thereby readying the device for another extraction operation. The operation handle may also be configured with a variable mechanical advantage, thus significantly reducing the input force required to be supplied by the user. The motion and minimal force necessary for performing an extraction allows the user to easily perform extraction operations using a single hand.

In a third embodiment of the invention, a first rotation of an operation handle pivots a gripping element about a spring-biased movable pivot axis, such that a gripping surface advances towards a stationary backing surface. With the needle cannula firmly held between the two surfaces, a continued rotation of the handle in the same direction pulls the needle cannula downwardly while the hub (or syringe tip) is retained outside (above) the orifice, causing the needle cannula to slide along the backing wall surface. By virtue of the spring-biased movable pivot axis, the needle cannula remains firmly gripped by the gripping surface throughout the extraction stroke, i.e., the stroke required to extract the needle from its associated hub or syringe tip. At the same time, binding or jamming of the mechanism is avoided and the extraction of needle cannulas of varying diameter is accommodated. The needle cannula may be reliably extracted from its associated hub or syringe tip, then released to fall harmlessly into an attached container.

In a fourth embodiment of the invention, a first rotation of an operation handle pivots a gripping element about a stationary pivot axis such that a gripping surface advances towards a spring-biased movable backing element to securely grip a needle cannula inserted therebetween. With the needle cannula firmly gripped, a continued rotation of the handle in the same direction causes the gripping element to pull the needle cannula downwardly, causing it to slide along the backing element. By virtue of the spring-biased movable mount of the backing element, a firm grip on needle cannulas of varying diameter can be maintained throughout an extraction stroke, without binding or jamming of the mechanism.

The above and other objects, features and advantages of the present invention will be readily apparent and filly understood from the following detailed description of preferred embodiments, taken in connection with the appended drawings.

### BRIEF DESCRIPTION OF THE DRAWINGS

FIG. 1 is an exploded perspective view of a first embodiment of a needle extraction device in accordance with the present invention.

FIG. 2 is a schematic cross-sectional view illustrating extraction elements of a needle extraction device of the type shown in FIG. 1.

FIGS. 3A-3F are schematic sectional views similar to FIG. 2, sequentially illustrating a needle extraction method in accordance with the present invention.

FIG. 4 is a side elevational view of a second needle extraction device according to the present invention.

FIG. 5 is a top plan view of the needle extraction device of FIG. 4.

FIG. 6 is an end perspective view of the needle extraction device of FIG. 4.

FIG. 7 is a bottom perspective view of the needle extraction device of FIG. 4.

FIG. 8 is a perspective view of a third needle extraction device in accordance with the present invention.

FIG. 9 is a top plan view of the device shown in FIG. 8, illustrating extraction elements therein.

FIG. 10 is a partially cut-away side elevational view, illustrating extraction elements of the needle extraction device shown in FIG. 8.

FIG. 11 is a proximal end elevational view of the needle extraction device shown in FIG. 8.

FIGS. 12-14 is are partial close-up sectional views sequentially illustrating a method of needle extraction utilizing the device of FIG. 8.

FIG. 15 is a perspective view of a fourth needle extraction device in accordance with the present invention.

FIG. 16 is a top plan view of the device shown in FIG. 15, illustrating extraction elements therein.

FIG. 17 is a side elevational view of the device shown in FIG. 15, illustrating extraction elements therein.

FIGS. 18-20 are partial side elevational views of the device shown in FIG. 15, sequentially illustrating a method of needle extraction utilizing the device of FIG. 15.

### DETAILED DESCRIPTION OF THE INVENTION

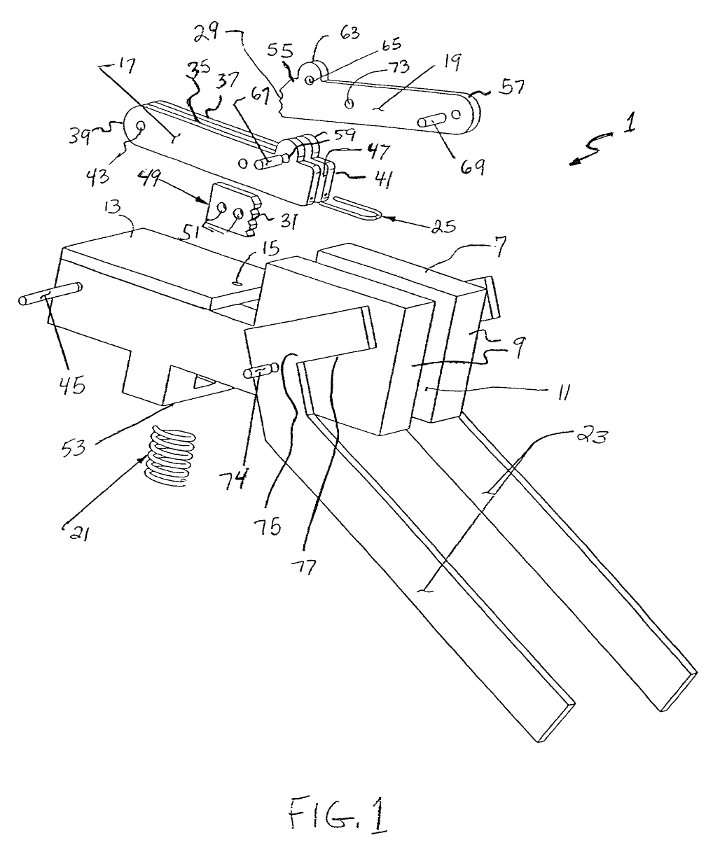

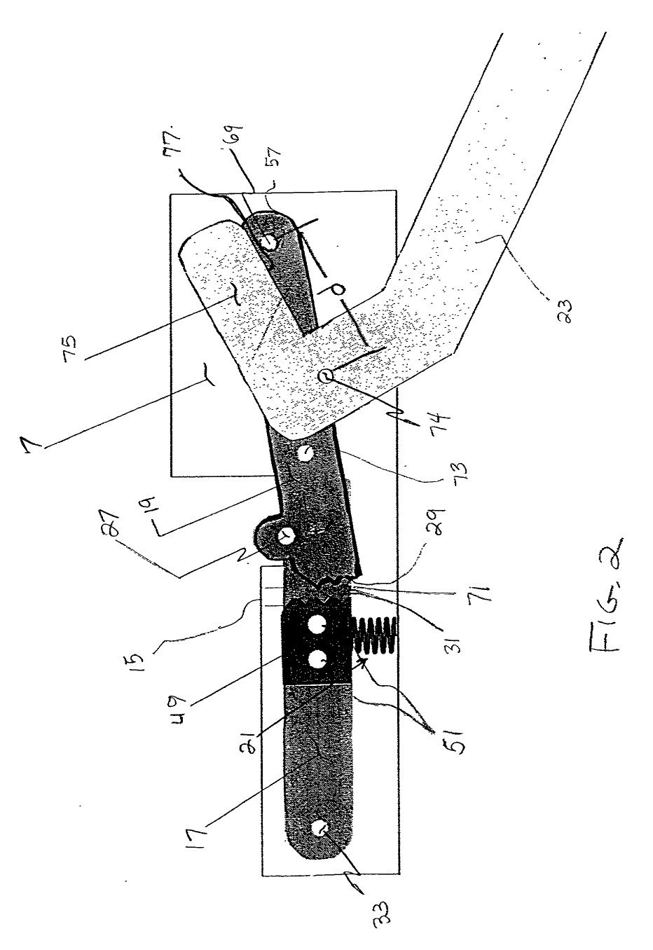

Referring to FIGS. 1-3, a first needle extraction device 1 in accordance with the present invention functions to reliably destructively extract a needle cannula 3 from its mounting hub 5 (or its direct mount to a syringe tip) by a compound rotary motion. The compound rotary motion serves to first engage an inserted needle, then to pull the needle with a firm grip on the needle which continues throughout an extraction stroke. As shown in FIGS. 1 and 2, needle extraction device 1 includes a body 7 which serves to house and operatively connect a plurality of extraction components. Although not shown in FIGS. 1-3, body 7 preferably also has an underside configured to provide a mount for an infectious sharps container serving to receive and retain extracted needles. Except as otherwise noted herein, the device elements may be constructed of various known plastic and/or metal materials, using conventional processes.

In the first illustrated embodiment, body 7 is formed by a pair of spaced side walls 9 defining a channel or slot 11 therebetween. A cover plate 13 is mounted across slot 11 and is affixed to distal top surfaces of side walls 9. Body 7 could alternatively be formed as a single molded or machined channel-defining component. Body 7 includes an orifice 15 extending through cover plate 13 and opening within channel 11. Orifice 15, which may be circular or of other shape, is made large enough to allow needle cannula 3 of various sizes to be inserted therein, but small enough to prevent hub 5 (or a syringe tip in the case of a direct syringe/needle attachment) from passing into the orifice.

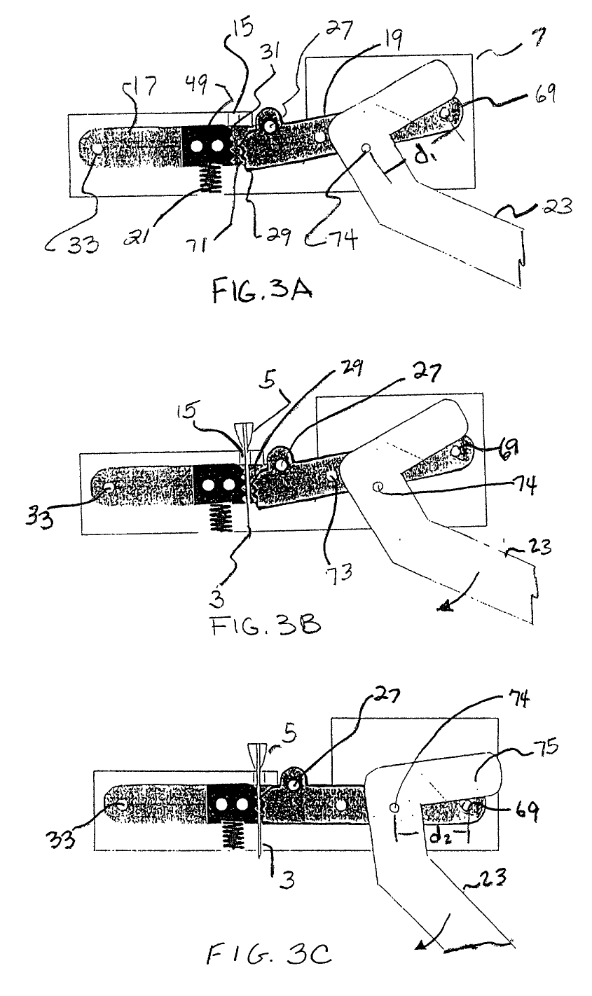

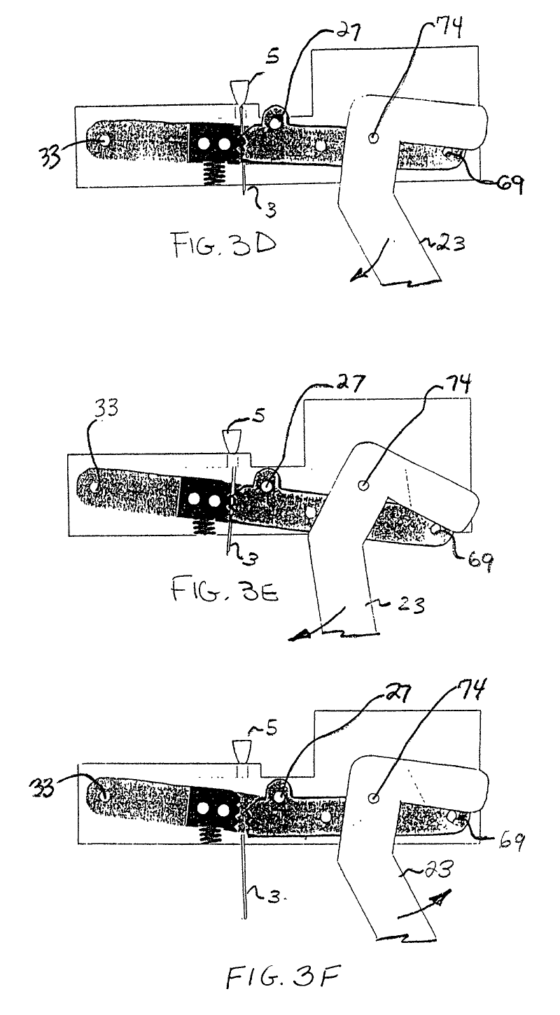

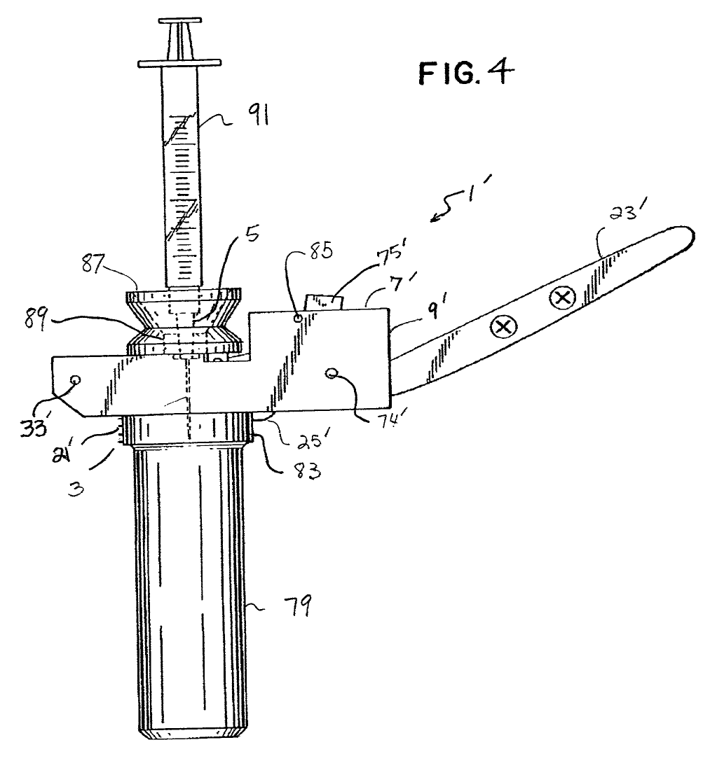

The operative extraction components of device 1 include a distal linkage 17, a proximal linkage 19, a distal linkage return biasing member (e.g., compression spring 21), an operation handle 23 and a proximal linkage biasing member 25. As mentioned, extraction device 1 extracts a needle cannula 3 inserted into orifice 15 from its hub 5 (or direct syringe mount) by a compound rotary motion, i.e., an initial needle engagement stroke and a subsequent needle extraction stroke. In the initial engagement stroke, a first rotation of proximal linkage 19 about a proximal pivot axis 27 (see FIGS. 2-3F), pivotably attaching proximal linkage 19 to distal linkage 17, moves a gripping surface 29 of proximal linkage 19 towards a gripping surface 31 of distal linkage 17. As a result, a needle cannula 3 inserted through orifice 15 is securely held between the two needle gripping surfaces 29, 31. At this point, distal linkage 17 and proximal linkage 19 engage with each other to rotate as a single extraction unit about a distal pivot axis 33 (see FIGS. 2-3F). While needle cannula 3 is firmly gripped, a continued rotation of operation handle 23 in the same direction (the extraction stroke) rotates the extraction unit about distal pivot axis 33 to forcibly separate needle cannula 3 from its hub 5. Thereafter, needle cannula 3 is released to fall harmlessly into an attached container (not shown in FIGS. 1-3F). Upon release of handle 23, spring 21 returns distal linkage 17 to its starting position, and return biasing member 25 returns proximal linkage 19, and handle 23, to their starting positions, whereupon gripping surfaces 29, 31 are separated from each other, making device 1 ready for another extraction operation. Spring 21 inherently serves to resist clockwise rotation of distal linkage 17 during the extraction stroke. As the resistive force of spring 21 is increased, so is the gripping force generated during the extraction stroke between gripping surfaces 29, 31. Thus, by adjustment of the biasing force of spring 21 (or an alternative biasing member), the needle cannula gripping force may be adjusted to a suitable level that maintains a firm non-severing grip on the needle throughout the extraction stroke.

As a variation on the above-described arrangement, gripping surfaces 29, 31 may be configured with profiles that cooperate to provide a “cam-lock” action serving to firmly grip the needle cannula. So arranged, once the “cam-lock” action has been effected, the needle cannula gripping force can be maintained independently of, or in cooperation with, the biasing force of spring 21.

As shown in FIG. 1, distal linkage 17 is constructed from two elongated bars 35, 37 disposed in spaced parallel relationship to each other, and having a distal end 39 and a proximal end 41. Bars 35, 37 include, adjacent distal end 39, aligned holes 43 permitting a pin 45 to extend through bars 35, 37 (and a spacer element positioned therebetween) and into corresponding receiving portions of body 7 to form distal pivot axis 33, about which the extraction unit formed by distal linkage 17 and proximal linkage 19 rotates during an extraction stroke.

The spacing of bars 35, 37 forms a slot 47 within which a gripping element 49, providing distal needle gripping surface 31, is fixedly mounted. Gripping element 49 is shown with a pair of holes 51 for screws or other fasteners to pass through to secure gripping element 49 between bars 35, 37. As an alternative to the illustrated multi-part structure, distal linkage 17 could be formed as a single elongated bar, with gripping element 49 mounted thereon or formed integrally therewith. Gripping element 49 provides a distal needle gripping surface 31 in the form of a serrated edge, for grasping needle cannula 3 in cooperation with a similar serrated edge of proximal gripping surface 29 provided on proximal linkage 19. Needle gripping surfaces 29, 31 should be formed of hardened steel or other materials harder than the metal of the needle cannulas to be extracted. Obviously, the construction of distal and proximal linkages 17, 19 could be reversed such that proximal linkage 19 comprises a slot-forming structure within which a single bar distal linkage 17 is pivotably mounted.

Return biasing member 21 (shown as a compression spring) serves to return distal linkage 17 to a generally horizontal (3:00) starting position after needle cannula 2 is extracted and released, as seen in FIG. 2. The upward swing of distal linkage 17, under the biasing force of spring 21, is restricted by cover plate 13. Return spring 21 is disposed within housing 7 underneath proximal end 41 of distal linkage 17, in abutting relationship with gripping element 49. As shown, spring 21 sits within a slot formed in a downwardly protruding portion 53 of body 7. Obviously, configurations other than the one illustrated may be used to provide an appropriate biasing force serving to return distal linkage 17 to its starting position, e.g., an appropriately mounted torsion or leaf spring.

Proximal linkage 19 comprises a single elongated bar having a distal end 55 and a proximal end 57. Proximal linkage 19 is pivotally mounted, adjacent its distal end 55, to distal linkage 17 via a pivot pin 67. As shown in FIG. 1, upper proximal edge portions of elongated bars 35, 37 include a pair of bosses 59 having aligned holes 61. The upper edge of linkage 19 has a similar boss 63, formed near its distal end 55, having a hole 65 formed therein. Proximal pivot axis 27 is formed by insertion of a pin 67 through holes 61, 65.

Serrated needle gripping surface 29 is formed at distal end 55 of proximal linkage 19. Obviously, the serrated edge can be formed as an integral part of end 55, or by attaching thereto a separately formed gripping element. Proximal end 57 of proximal linkage 19 includes a pin 69 extending transversely therethrough, thereby forming a pair of arms protruding from respective opposite sides of linkage 19. The protruding arms form part of a sliding pivotal connection with operation handle 23, to thereby provide a variable mechanical advantage system to be described.

As can be seen in FIG. 2, proximal linkage 19 is initially pivoted on proximal pivot axis 27 to a small angle of inclination (e.g., 10° degrees), as measured relative to the generally horizontal (3:00) orientation of distal linkage 17, so as to create a small passageway-forming gap 71 between the two needle gripping surfaces 29,31. Return biasing member 25 biases proximal linkage 19 into this initial position. It will be appreciated that the initial inclination angle can be any angle that provides clearance sufficient for needle cannula 3 to be inserted between gripping surfaces 29, 31, e.g., from 5° to 45° with respect to the initial 3:00 orientation of distal linkage 17. Preferably, the initial inclination angle is minimized within the foregoing constraint, in order to shorten the initial needle engagement stroke.

Biasing member 25 can be of any type serving to bias proximal linkage 22 to its initial inclined position, such as a compression spring. In the embodiment shown, return biasing member 25 is a spring formed of spring (piano) wire bent into a “U” shape. The two legs of the U-shape are anchored in holes provided in proximal ends 41 of distal linkage bars 35, 37 respectively. The apex of the U-shape is bridled within a hole 73 provided in proximal linkage 22. The U-shaped wire is resiliently bent to create a biasing force which increases as proximal linkage 19 is rotated downwardly.

Operation handle 23 is pivotally attached to body 7, within slot 11, by a pin 74. As depicted in FIG. 1, handle 23 can be constructed from two like, generally J-shaped, arms fastened together in parallel relation by any appropriate method, such as screws, adhesives, etc. Handle 23 includes a lever arm 75 which provides an increased extraction force by way of variable mechanical leverage. In particular, the geometry of lever arm 75 causes arm-forming pin 69 to slidably engage an interior edge 77 of lever arm 75. (Alternatively, pin 69 could ride within a slot formed in lever arm 75.) Referring to FIG. 2, an effective lever having a distance d is created between pin 69 (the point of engagement of arm 75 with proximal linkage 19) and pin 74 (the pivot axis of handle 23). As handle 23 is rotated downwardly, a downward force is applied to pin 69 by lever arm 75. As a result, lever arm 75 pushes pin 69 and attached proximal linkage 19 downward. The downward force applied to pin 69 is proportional to the user's input force, and the effective lever distance d. As effective lever distance d becomes shorter, the downward force applied to pin 69 increases. As handle 23 is rotated downwardly in the initial engagement stroke, the sliding action of pin 69 along surface 27 causes pin 69 to move towards pin 74. As a result, the effective lever distance d is shortened and the resultant downward force acting on pin 69 increases to a maximum at initiation of the extraction stroke. During continued rotation within the extraction stroke, pin 69 is caused to move back toward its initial position, decreasing the resultant force on pin 69, but increasing the angular displacement of proximal linkage 19 relative to the angular displacement of handle 23.

FIGS. 3A-3E illustrates an operational sequence of a needle extraction method in accordance with the present invention. As shown in FIG. 3A, extraction device 1 is in a starting position. In this starting position, proximal linkage 19 is biased by biasing member 25 (not shown) to a small inclination relative to distal linkage 19, to thereby create passageway 71 between needle gripping surfaces 29, 31. Upon being inserted into orifice 15, a needle cannula may pass into passageway 71. Return biasing member (spring) 21 holds distal linkage 19 generally horizontal between the top and bottom surfaces of body 7.

In FIG. 3B, a needle cannula 2 has been inserted into orifice 15 and passed into passageway 71 formed between gripping surfaces 29, 31. Next, the user grips handle 23 and rotates the same (downwardly and clockwise as shown) against the relatively small biasing force of biasing member 25. In the initial needle engagement stroke, proximal linkage 19 rotates solely about proximal pivot axis 27. As the handle is rotated, gripping surface 29, provided at the distal end 55 of proximal linkage 19, pivots toward gripping surface 31 of distal linkage 17, to securely grip needle cannula 3 therebetween. From this point forward, linkages 17, 19 are engaged to rotate through an extraction stroke as a rigid unit.

Further downward rotation of handle 23, from the position shown in FIG. 3C, causes engaged linkages 17, 19 to rotate as a unit clockwise about distal pivot axis 33. At this point, needle cannula 3 begins to be pulled downwardly, while needle hub 5 is retained outside of orifice 15 by a surrounding surface area of cover plate 13. Then, as shown in FIGS. 3D and 3E, the user continues to rotate handle 26 downwardly, which rotates linkages 17, 19 as a unit downwardly against the relatively small upward bias of return spring 21, and separation of needle cannula 3 from its hub 5 is initiated. As rotation continues through this extraction stroke, and as shown in FIG. 3E, needle cannula 3 is eventually fully separated from its hub 5 while remaining securely captured between needle gripping surfaces 29, 31.

As depicted in FIG. 3F, release of needle cannula 3 can be effected by the user releasing or otherwise returning handle 23 to its starting position shown in FIG. 3A. Upon release or return of handle 23, the bias of return spring 21 will return distal linkage 17 to its initial 3:00 position, and proximal linkage 19 will return to its initial slightly inclined position, thus readying device 1 for another removal operation.

It should be noted that in FIG. 3C, the effective lever distance d is reduced to d2, from the initial distance d1 shown in FIG. 3A. By way of the resultant variable mechanical advantage, the angular handle displacement (engagement stroke) necessary to effect gripping of needle 3 is reduced, while an increased extraction force is obtained at initiation of the extraction stroke. As the extraction stroke is continued, as shown in FIGS. 3D-3F, the effective lever distance d increases from d2. As a result, the distance moved increases while the applied downward force decreases. This advantageously approximates the force profile required for needle removal. One of ordinary skill in the art will appreciate that by changing the position of pin 69 relative to handle pivot pin 74, the maximum downward force applied to pin 69 for a given input force can be varied. Further, the geometry of the pin engaging interior edge 77 (or slot) can also be changed to create a variety of force profiles. It should also be noted that the invention contemplates needle extraction devices lacking an operation handle 23, configured as shown, to provide a variable mechanical advantage. Instead, a simpler arrangement within the scope of the invention would employ a handle formed as a simple continuation of proximal linkage 19, extending proximally from body 7 and having of a known type of hand grip thereon. Such an arrangement would permit proximal linkage 19 to be pivoted directly by hand.

Devices according to the present invention permit extraction of a wide range of needle sizes by persons with minimal hand strength. For example, the force required to pull a needle from its hub may range from a low of several pounds for a 28-gauge needle, to over 70 lbs. for a 16-gauge needle. With the present inventive device, a 16-gauge needle can be removed by application of a hand force within the range of approximately 12-16 lbs. In addition, the device of the invention can be operated with one hand without complicated movements.

FIGS. 4-7 illustrate an operative prototype needle extraction device 10′ according to the present invention, assembled from components machined from aluminum stock. It will be appreciated that a commercial embodiment likely would be constructed differently, e.g., from molded and assembled plastic parts (except for the needle gripping surfaces, that should be formed of hardened steel or other material harder than the needles to be pulled). Prototype needle extraction device 1′ includes a body 7′ and attached needle container 79. Body 7′ houses and operatively mounts extraction components essentially as illustrated in FIGS. 1-2, and operates in essentially the manner shown in FIGS. 3A-3F.

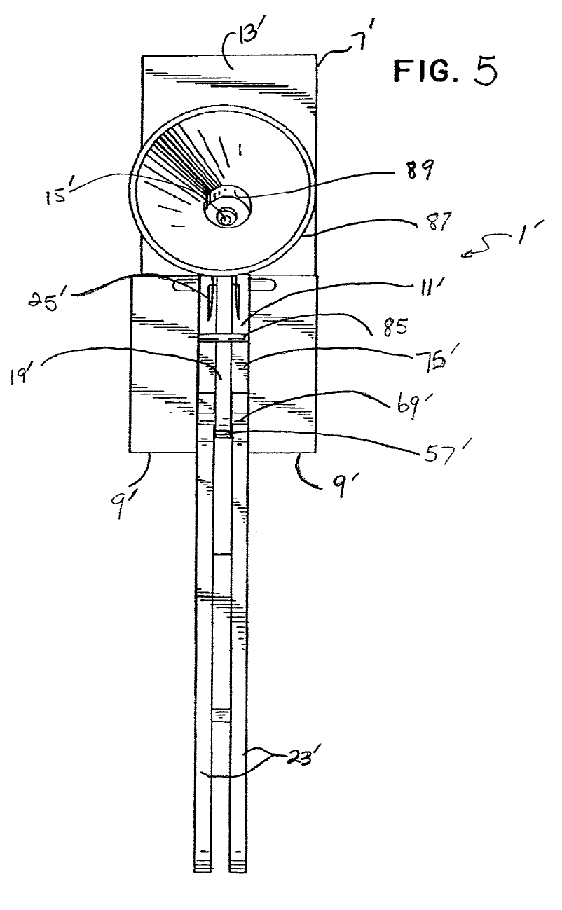

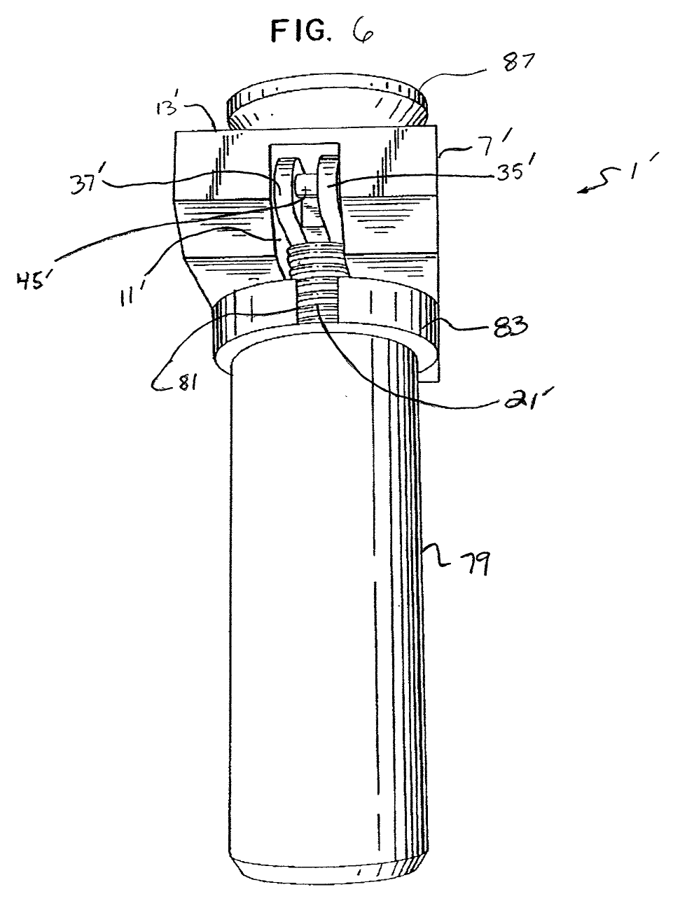

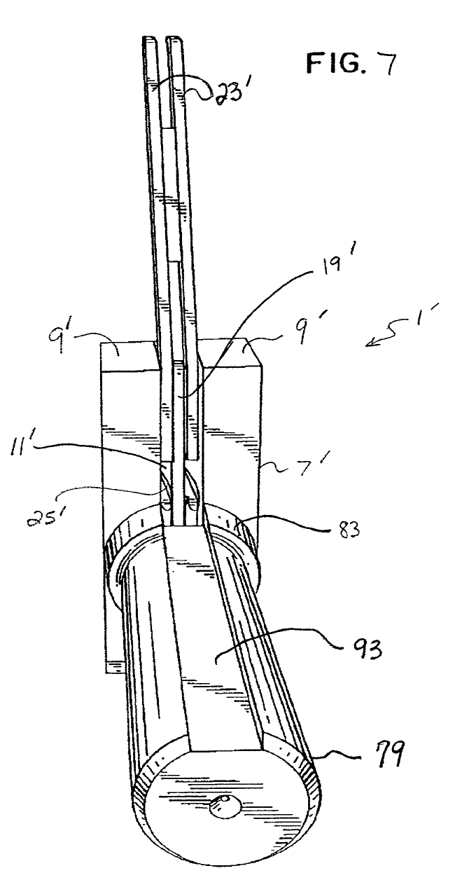

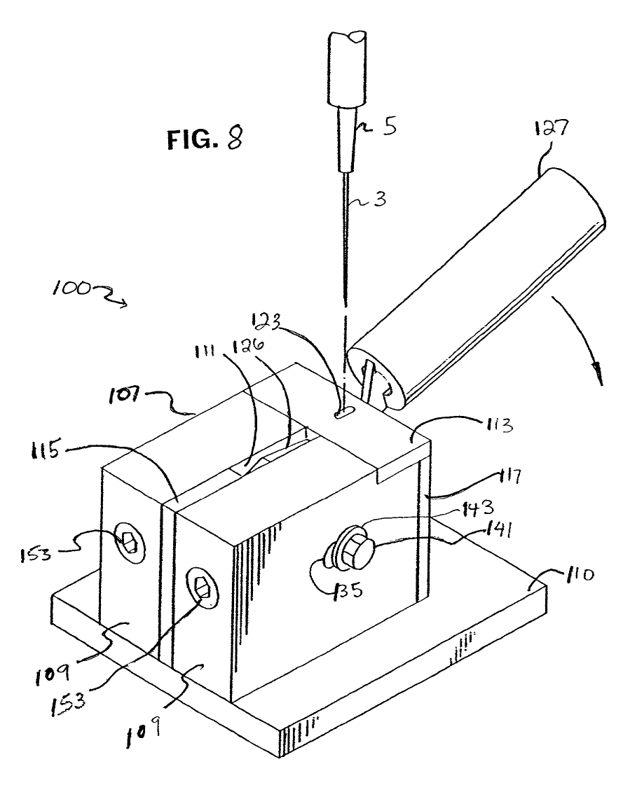

As seen in FIG. 6, distal linkage return spring 21′ is retained in a notch 81 provided in an upper circular flanged portion 83 of container 79. With reference to FIGS. 4 and 7, operation handle 23′ is shown pivoted upwardly to an inclined starting position by the biasing force of a U-shaped piano wire spring 25′. Referring to FIG. 5, it can be seen that body 7′ further includes a stop bar 85 spanning the slot 11′ formed between side walls 9′, adjacent the top surface of body 7′, for limiting the upward angular travel of handle 23′ under the biasing force of spring 25′.

Referring to FIGS. 4-5, it is seen that an entry cone or funnel 87 is mounted at the top portion of body 7′ to facilitate insertion of a needle cannula 3 into orifice 15′. An internal shoulder portion 89 of entry cone 87 serves to position and maintain a syringe 91 in a generally upright orientation, as seen in FIG. 4, thereby allowing one-handed operation.

Container 79 serves to receive and store extracted contaminated needles, and also provides a convenient hand grip against which operation handle 23 may be squeezed. As shown, a lengthwise slot 93 is provided in the cylindrical side wall of container 79 to provide additional clearance for downward rotation of handle 23′. Such a slot can also be used to flushly receive handle 23′ at the end of its stroke. Container 79 may attach to the bottom of body 7′ by various known means, such as a threaded engagement permitting container 79 to be mounted on, and removed from, body 7′ by manual axial rotation, without the need for tools. Container 80 obviously may be embodied in many other forms and be constructed of various known materials.

FIGS. 8-14 show a third embodiment of the invention in the form of a needle extraction device 100. Device 100 functions to reliably destructively extract a needle cannula 3 from its mounting hub 5 (or its direct mount to a syringe tip) by rotating a gripping surface toward a stationary backing surface about a spring-biased movable pivot axis, to firmly and continuously grip an inserted needle throughout an extraction stroke. As will be described, movement of the pivot axis (perpendicular to its extending direction) during the extraction stroke allows a firm grip on needle cannulas of varying thickness (e.g., diameter in the case of a shaft of circular cross-section) to be maintained, while avoiding binding or jamming of the mechanism.

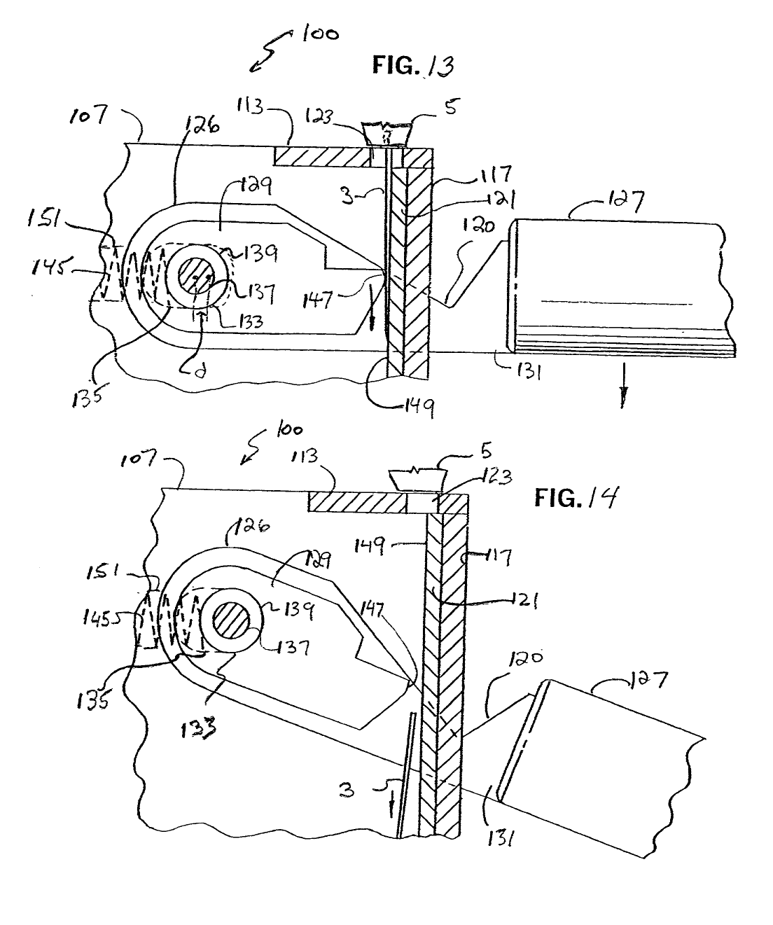

Needle extraction device 100 includes a plurality of elements that may be constructed of various known plastic and/or metal materials, using conventional processes. A body 107 of device 100 comprises a pair of side wall blocks 109 secured on a base plate 110. Blocks 109 extend parallel to each other and define a channel 111 therebetween. An abbreviated top plate 113 is affixed to recessed proximal top surfaces of side wall blocks 109 and extends across slot 111. A spacer plate 115 is mounted between side wall blocks 109 at a distal end of body 107. A side plate 117 is secured to, and covers, the proximal ends of blocks 109. As seen in FIG. 11, side plate 117 has a vertical slot 119 which opens into channel 111. The slot is made about half the width of channel 111, such that an inside of plate 117 forms a narrow vertical ledge on which may be secured a stationary backing plate 121. Instead of the illustrated multi-part construction, body 107 obviously could be formed as a unitary molded or machined piece, of like form.

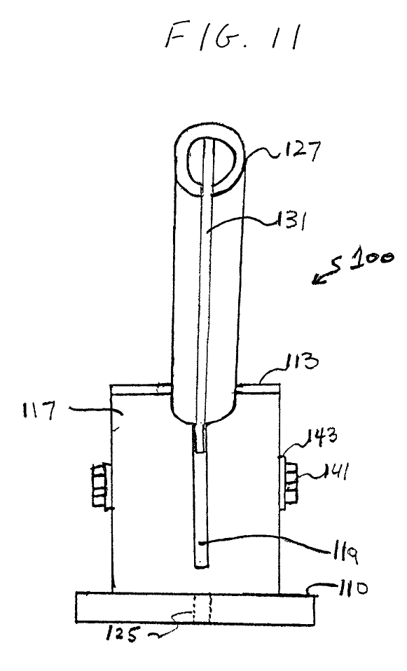

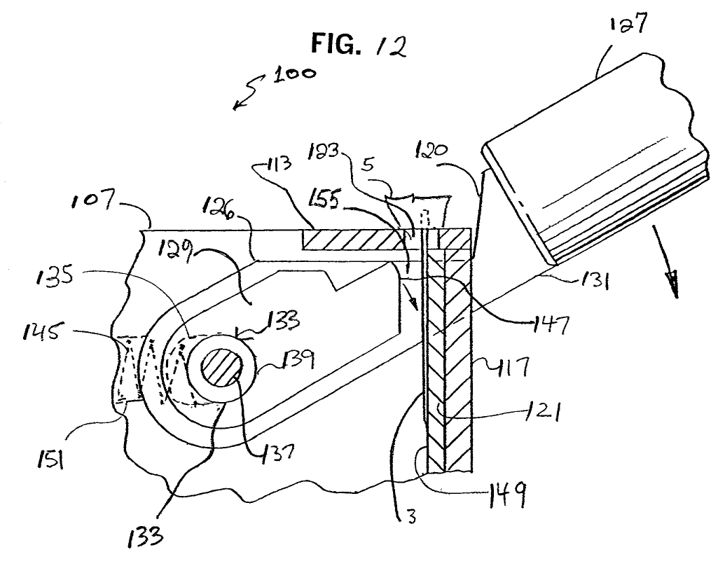

An orifice 123 extends through top plate 113 and opens into channel 111. Orifice 123, which may be circular, slightly elongated, or of other shape, is made large enough to allow needle cannula 3 of various sizes to be inserted therein, but small enough to prevent hub 5 (or a syringe tip in the case of a direct syringe/needle attachment) from passing into orifice 123 (see FIG. 12). As depicted in FIG. 10, base plate 110 can have a through passage 125. An underside of base plate 110 may be configured to provide a mount for an infectious sharps container, for receiving and retaining extracted needles dropped through passage 125.

Device 100 includes a lever 126 which is provided at its proximal end with a handle 127. Mounted at a distal end of lever 126 is a gripping element 129. As shown, lever 126 is constructed as an elongated flat metal bar 131, and handle 127 is formed as an elongated tubular member mounted on bar 131. A distal end of lever 126 is pivotally mounted on a pivot pin/sleeve combination 133 which is movable proximally and distally within a pair of slots 135 provided in wall blocks 109. As best seen in FIG. 12, the pivot pin/sleeve combination comprises a pivot pin 137 which extends within a tubular sleeve 139. Pin 137 has threaded ends which engage lock nuts 141 maintained to the sides of the slots 135 by respective washers 143 (See FIG. 8). Sleeve 139 serves as a bushing for mounting pin 137 in slots 135, and to provide a low-friction bearing surface upon which lever 126 may rotate. Obviously, a pivot axis of lever 126 could be configured otherwise, such as with a bare pivot pin (lacking a sleeve), serving as a direct pivotal mount of lever 126. Pivot pin/sleeve combination 133 is biased to (or toward) the proximal ends of slots 135 by a pair of biasing members, e.g., springs 145. The ends of slots 135, or other suitable structure, may serve as a stop limiting movement of the pivot pin/sleeve combination 133 under the biasing force of springs 145, to establish a suitable rest position. Lever 126 passes out of channel 111 through aligned slot 119 provided in side plate 117. Slot 119 is of a length providing a clearance which allows lever 126 to pivot through an angular displacement of approximately 60°, from a top position abutting a proximal edge of top plate 113 to a bottom position abutting with a top surface base plate 110. A generally V-shaped notch or cut-out 120 is provided in an upper edge of lever 126 to afford additional clearance enlarging the pivotal throw of lever 126 within slot 119.

Gripping element 129 can be formed as a plate of metal or other relatively hard material fixedly attached to a flat distal surface of lever 126 by any suitable method, e.g., adhesive bonding, welding or mechanical fasteners. Alternatively, a gripping element could be formed integrally as a single unit with lever 126. A gripping surface 147 is provided on a proximal edge of element 129. The proximal edge tapers distally from a point slightly below a top edge of element 129 to a bottom edge thereof, thereby forming needle gripping surface 147 as a blunted point. This taper provides a clearance permitting gripping element 129 to pivot freely into contact with an inserted needle cannula. As illustrated, gripping surface 147 is generally smooth. Alternatively, needle gripping surface 147 may comprise a serrated, knurled or otherwise textured edge. To avoid excessive wear, the needle gripping surfaces are preferably formed of hardened steel or other material at least as hard as the material of the needles to be pulled.

A distal end of lever 126 has a hole aligned with a hole provided in gripping element 129. Pivot pin/sleeve combination 133 extends through these aligned holes and into corresponding slots 135 to form a moveable pivot axis. This arrangement permits simultaneous movement of the pivot axis in the proximal and distal directions, and slidable rotation of lever 126 (including distal gripping element 129) on pivot pin/sleeve combination 133 during an extraction operation.

Backing surface 149 can be provided by a plate 121 of steel or other relatively hard material fixedly mounted to an inside of plate 117, so as to cover the narrow vertical ledge area in direct opposition to gripping surface 147. Backing surface 149 should be smooth and low friction, so that needle cannula 3 slides easily therealong during an extraction operation.

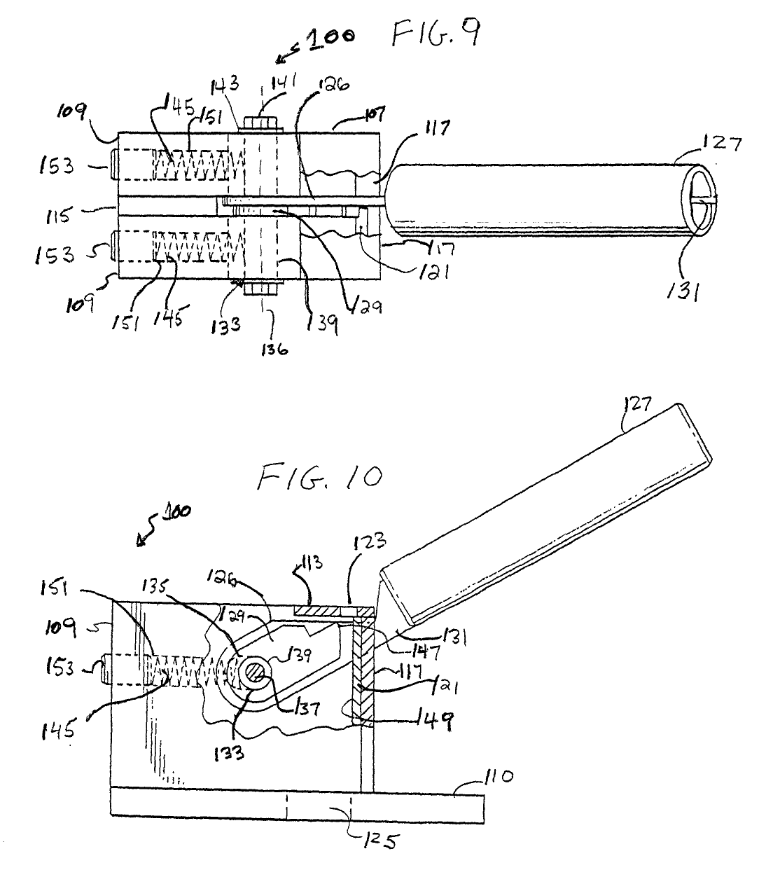

Springs 145 serve to apply a continuous, proximally directed, biasing force on pivot pin/sleeve combination 133. As can best be seen in FIG. 9, proximal ends of springs 145 abut against sleeve 139, forcing pivot pin/sleeve combination 133 to (or toward) the proximal end of slots 135. As shown, springs 145 are helical compression springs disposed inside of respective cylindrical passages 151 formed within respective side wall blocks 109. Springs 145 are compressed within passages 151, between pin/sleeve combination 133 and respective stoppers 153 threadably received in the ends of passages 151. The stoppers can be threadably retracted or advanced within passages 157 to adjust the amount of compression of springs 145, and hence the biasing force exerted on the pivot pin/sleeve combination.

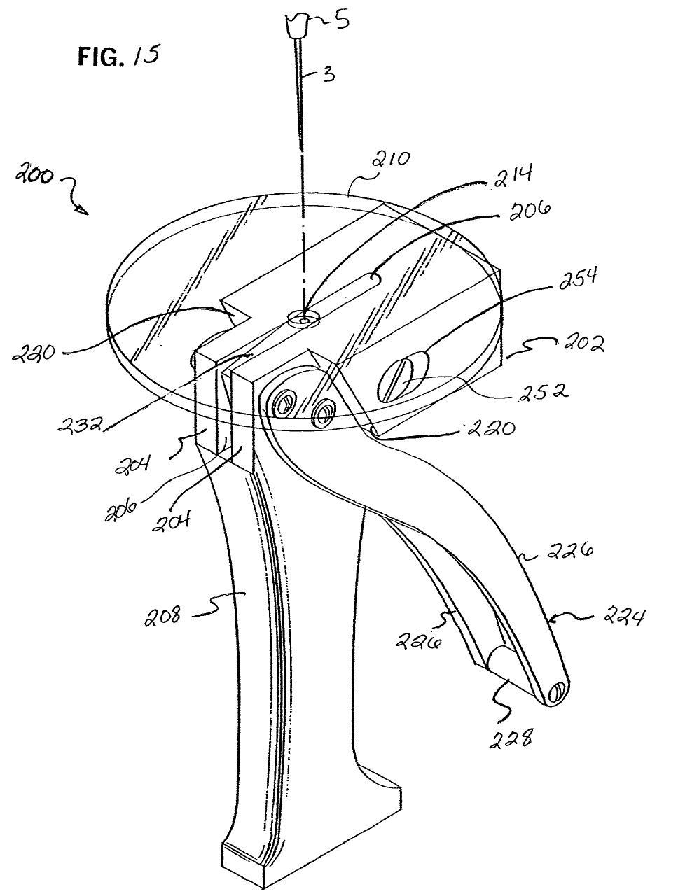

FIGS. 12-14 illustrate an operational sequence of a needle extraction method in accordance with the present invention, as carried out with extraction device 100. As shown in FIG. 12, device 100 is in a starting position and a needle cannula 3 has been inserted into orifice 123 such that hub 5 abuts with a surrounding surface of plate 113. In this starting position, handle 127 is initially positioned in a slightly upwardly inclined (e.g., 2:00) orientation, providing a passageway-forming gap 155 between gripping surface 147 and backing surface 149, within which needle cannula 3 extends. Next, the operator grips handle 127 and rotates the same downwardly (clockwise as depicted). As the handle is rotated, gripping surface 147 pivots towards backing surface 149. Needle cannula 3 is securely gripped by surface 147 and is pressed against backing surface 149, as shown in FIG. 13. As the handle rotation continues, gripping surface 147 grips and pulls downwardly on needle cannula 3, causing needle 3 to slide downwardly along backing surface 149 and initiating separation of needle 3 from its hub 5. At the same time, gripping surface 147 is pressed with increasing force against backing surface 149. Eventually, the biasing force of springs 145 on pivot pin/sleeve combination 133 is overcome, at which point pivot pin/sleeve combination 133 moves distally within slots 135. As depicted in FIG. 13, pivot pin 137 moves distally a distance “d” generally equal to the diameter of needle cannula 3 at the engagement point of gripping surface 147. The distal movement of pivot pin 137, against the bias of springs 145, permits needle gripping surface 147 to maintain a firm grip on needle cannula 3 throughout an extraction stroke, while avoiding binding or jamming of needle cannula 3 against backing surface 149. As can be appreciated, further downward rotation of handle 127, from the center (horizontal) position shown in FIG. 13, causes needle cannula 3 to continue to be pulled downwardly (slidably against backing surface 149), while needle hub 5 is retained outside of orifice 123. Needle cannula 3 is eventually fully separated from its hub 5, while remaining captured between needle gripping surface 147 and backing surface 149.

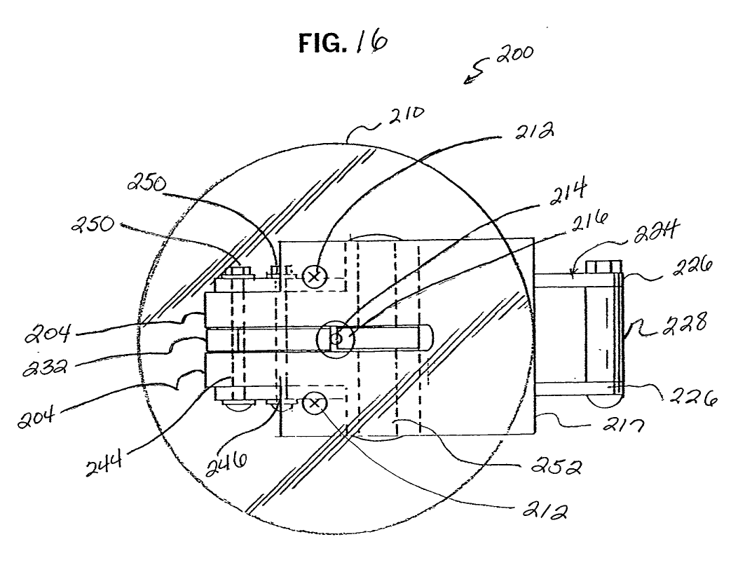

Then, as depicted in FIG. 14, release of needle cannula 3 is effected by the operator continuing downward handle rotation. Once gripping surface 147 has passed the center position shown in FIG. 13, further rotation of handle 127 begins to withdraw gripping surface 147 from backing surface 149 and thereby reduces the force exerted by gripping surface 147 on needle cannula 3 (and backing surface 149). As this force is reduced below the sum of the biasing forces exerted by springs 145 on pivot pin/sleeve combination 133, the latter makes a return trip to its initial position at the proximal ends of slots 135. As shown in FIG. 14, eventually gripping surface 147 separates from the now extracted needle cannula 3, allowing the needle cannula to fall freely through discharge chute 125 (see FIG. 10) for disposal, e.g., into an attached sharps container (not shown). The user can then return handle 127 to its initial (2:00) position, thus readying device 100 for another removal operation. Such return action may alternatively be achieved automatically by spring-biasing lever 126 to its starting position.

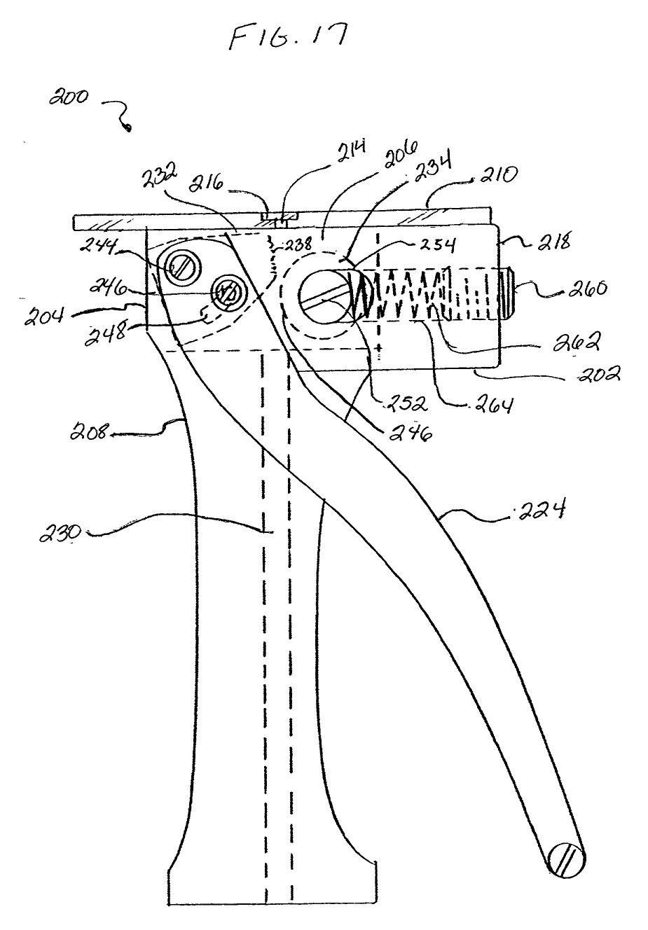

FIGS. 15-20 illustrate a fourth exemplary needle extraction device 200 according to the present invention, for reliably and destructively extracting a needle cannula 3 from its mounting hub 5, by way of pivoting a lever having at its upper end a gripping surface that advances towards a backing member, to firmly grip an inserted needle throughout an extraction stroke. In this embodiment, it is the backing member, rather than the pivoted gripping element, that is mounted for spring-biased translational movement in the proximal and distal directions.

Device 200 includes a body 202 which may be formed as a single molded or machined component. Body 202 has at its upper end a pair of spaced parallel sidewalls 204 defining a channel 206 within which extraction components are operationally mounted. Below sidewalls 204 is formed a contoured pistol-style hand grip 208. A hand shield 210 is affixed to the top surface of the body 202 by screws 212 (see FIG. 16), adhesive or the like. Shield 210 serves to protect an operator's hand from needlestick injury during use of device 200. As shown, shield 210 is circular and made of transparent plastic to reveal the operative extraction component within channel 206. Obviously, shield 210 may be made of alternative shapes and materials, including a funnel-shape to guide needle cannula 3 into orifice 214.

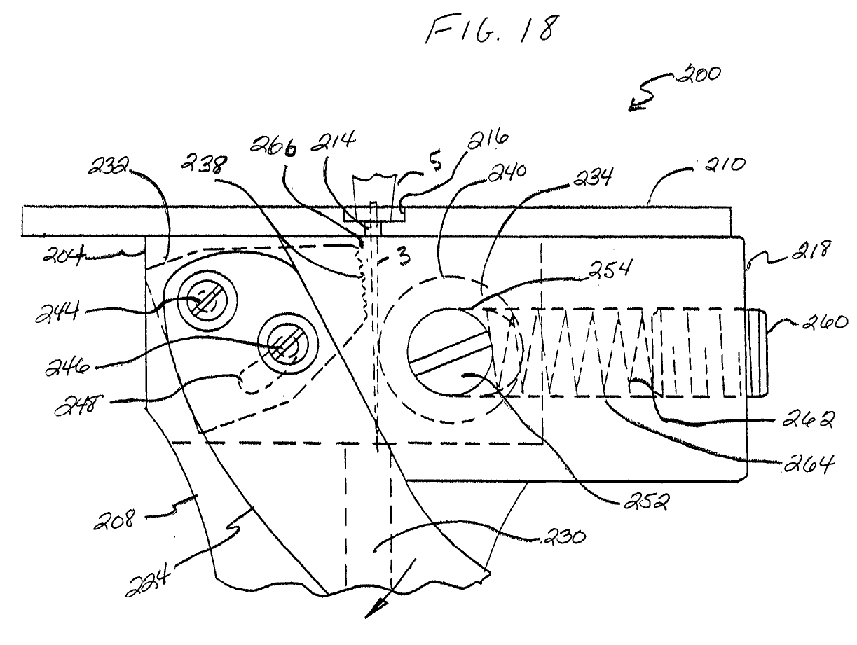

Orifice 214 extends through shield 210 and opens above channel 206. Orifice 214, which may be circular, or of other shape, is made large enough to allow needle cannulas 3 of various sizes to be inserted therein, but small enough to prevent hub 5 from passing into orifice 234. As seen in FIGS. 17 and 18, orifice 214 is situated within a recess 216 serving to assist with needle insertion and in axial alignment of an associated syringe in a perpendicular orientation with respect to shield 210. As an alternative to the illustrated counter-bore recess 216, a conically shaped or otherwise tapered recess may be utilized to further assist with guiding a needle cannula 3 into orifice 214.

Body 202 includes a block portion 218 cantilevered from the top end of pistol grip 208 and forming a tapered ridge 220 relative to inset sidewalls 204. Channel 206 formed between sidewall 204 extends uninterruptedly into block portion 218, through a central part thereof. Inset sidewalls 204 provide a mounting location for a pivot pin 244 upon which an operation handle/lever 224 is pivotably mounted. As shown in FIG. 15, lever 224 can be constructed from two elongated flat bars 226 fastened together in spaced parallel relation, separated at their upper ends by channel-forming sidewalls 204, and at their lower ends by a tubular spacer 228 which is secured by a bolt extended through bars 226 and spacer 228.

Hand grip 208 allows an operator to hold device 200 and simultaneously squeeze lever 224 toward grip 208, for performing an extraction operation with one hand. As depicted in FIG. 17, an elongated passage 230 is included inside of hand grip 208, through which extracted needle cannulas may fall, for deposit into a sharps waste container (not shown). If desired, hand grip 208 can be made hollow or otherwise configured to serve, itself, as a sharps container serving to receive and store extracted needles. Alternatively, a separate container may be attached to, or otherwise positioned below, the underside of grip 208.

Referring to FIGS. 17-20, lever 224 has mounted at its proximal end, and within channel 206, a gripping element 232. A backing element 234 is mounted inside channel 206 in opposing alignment with gripping element 232. Backing element 234 is mounted for translational movement, proximally and distally, on a spring-biased movable pin 252.

Gripping element 232 is constructed from a small metal plate having a gripping surface 238 formed on a protruding free edge thereof. As shown, gripping surface 238 comprises a serrated edge. The upper ends of lever bars 226 are mounted to sidewalls 204 by a pivot pin 244 extending through the sidewalls and through gripping element 232. As seen in FIGS. 17-20, a pin 246, offset from pivot pin 244, also extends through lever bars 226 and gripping element 232. Pin 246 passes through arcuate slots 248 formed in sidewalls 204. Pin 246 rides within slots 248 as handle 224 is rotated, and functions to provide additional mechanical advantage in rotating gripping element 232 as lever 224 is rotated about pivot pin 244. Slots 248 also serve to limit the downward swing of pin 246, thereby serving to prevent rotation of lever 224 beyond grip 208. Rotation of lever 224 in an opposite direction (counterclockwise as depicted) may be limited by slots 248 and/or the tapered ridge 220 formed between block portion 218 and inset sidewalls 204. Pivot pins 244 and 246 may be formed by screws secured by lock nuts 250 (see FIG. 16), or the like.

Referring to FIG. 17, backing member 234 comprises a circular disk concentrically rotatably mounted on a pin 252. Pin 252 is movable proximally and distally within slots 254 formed to each side of channel 206 in block portion 218, and is biased to proximal ends of slots 254 by a pair of helical compression springs 262. As with the third embodiment, other biasing members may be utilized.

Backing member 234 includes an outer circumferential surface 240 which serves, together with gripping surface 238, to grip therebetween an inserted needle cannula 3. As shown, outer surface 240 is smooth; it may, alternatively, be serrated or otherwise textured. Similar to device 100, springs 262 function to provide a continuous biasing force on a pivot axis of a needle contacting member, to prevent jamming and binding of the extraction mechanism. In this case, however, the member which is made moveable under spring bias is backing member 234, which is rotatable on pivot pin 252. Springs 262 are disposed inside of cylindrical passages 264 formed within block portion 218, to each side of channel 206. Similar to the first embodiment, springs 262 are compressed within cylindrical passages 264 between pin 252 and respective stoppers 260 threadably received in the ends of the passages; the position of the stoppers is thereby adjustable to adjust the compression, and hence the biasing force, of springs 262.

FIGS. 18-20 illustrate an operational sequence of a needle extraction method in accordance with the present invention, carried out with device 200. As shown in FIG. 18, device 200 is in a starting position and a needle cannula 3 has been inserted into orifice 214 such that hub 5 abuts with a surrounding surface of shield 210. In this starting position, with lever 224 positioned generally in a 5:00 orientation, a gap 266 is formed between gripping surfaces 238, 240. Upon insertion, needle cannula 3 extends through gap 266. Next, the operator grips lever 224 and hand grip 208, and squeezes the two together. As lever 224 is rotated about pin 244, outer surface 238 pivots towards needle cannula 3 and forces the needle against gripping surface 240 of backing member 234.

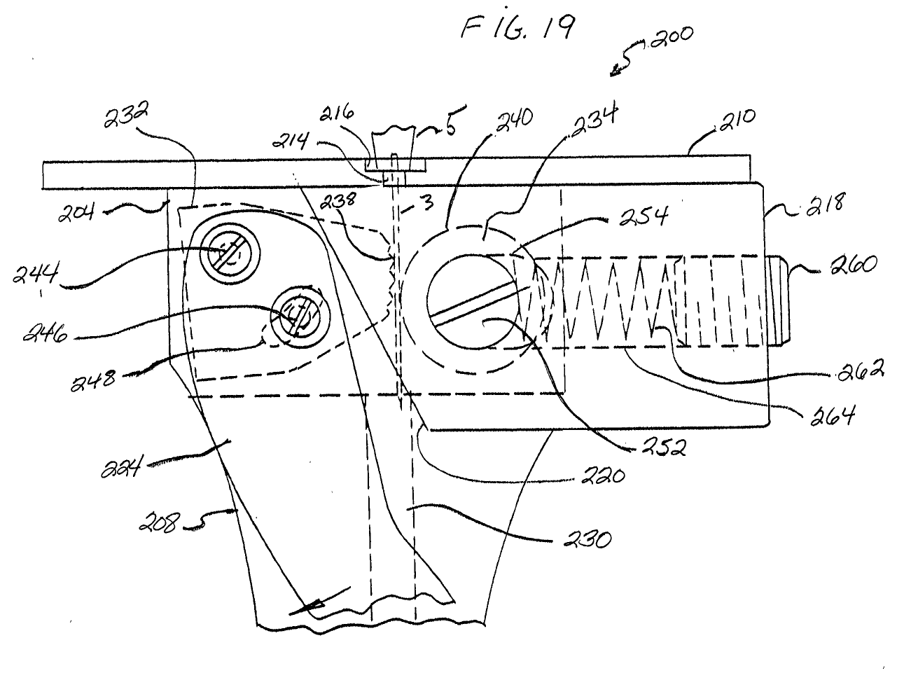

In FIG. 19, needle cannula 3 has been engaged between gripping surface 238 and surface 240. As the lever rotation continues, gripping surfaces 238 and 240 grip and pull downwardly on needle cannula 3 with a cooperative rolling motion, thus initiating separation of needle 3 from its hub 5. At the same time that gripping element 232 is rotating clockwise (as depicted) about pin 244, backing member 234 rotates counterclockwise about pin 252. Alternatively, backing member 234 could be made non-rotatable, in which case, needle cannula 3 would be engaged by gripping surface 238 and slid along a stationary contact surface, similar to the second embodiment.

Similar to the operation of the third embodiment, gripping surface 238 is pressed with increasing force against backing member 234 to the point that the biasing force of springs 262 on pin 252 is overcome, at which point pin 252 moves distally within slots 254. The distal movement of pin 252 permits gripping surfaces 238 and 240 to maintain a firm grip on needle cannula 3, while avoiding binding or jamming of the needle cannula therebetween. Further downward rotation of lever 224 toward hand grip 208 causes needle cannula 3 to be pulled further between gripping surfaces 238, 240, while needle hub 5 is retained outside of orifice 214 by the surrounding portion of shield 210. Needle cannula 3 is eventually fully separated from its hub 5 while remaining securely captured between gripping surface 238 and surface 240.

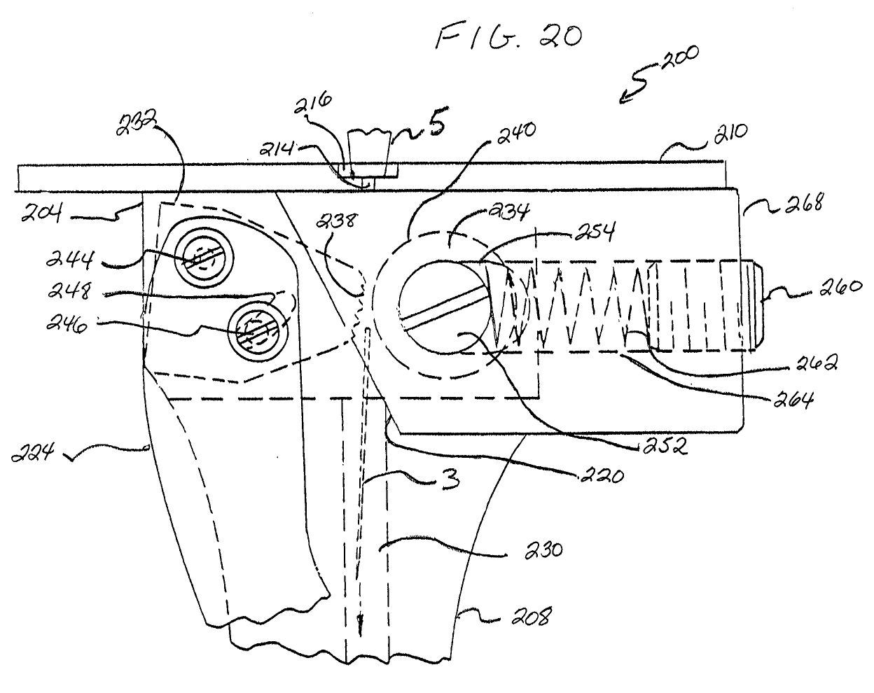

As shown in FIG. 20, needle cannula 3 is released upon further rotation of lever 224. Such further rotation beyond the point of maximum advancement of gripping surface 238 toward gripping surface 240 reduces the force exerted between backing member 234 and gripping element 232. As this force is reduced below the biasing force of springs 262, pin 252 begins a return trip to its initial position at the proximal end of slots 254. Eventually, gripping surface 238 separates from the now extracted needle cannula 3, allowing the needle cannula to fall through passage 230 and harmlessly into a sharps container (not shown). The user can then return lever 224 to its initial position, thus readying device 200 for another extraction operation. Such return action may alternatively be achieved automatically by spring-biasing lever 224 to its starting position.

The present invention has been described in terms of exemplary embodiments thereof. Numerous other embodiments, modifications and variations within the scope of the invention may occur to persons of ordinary skill in the art from a review of this disclosure. For example, as a variation on the third embodiment illustrated in FIGS. 8-15, the operative components may be rearranged and reconfigured such that the pivoting needle gripping surface faces distally (e.g., at a distal end of the pivotable bar), and the opposed backing surface is positioned distally of the gripping surface (facing proximally). With such an arrangement, a needle cannula extraction stroke would be carried out with an upward (rather than downward) rotation of the handle. Also, either of the needle gripping surface or the backing surface could be mounted for spring biased movement, in accordance with the principals described, respectively, in connection with the second and third embodiments. As another example of a variation within the scope of the invention, any of the embodiments could receive input force by way of powered means such as electric solenoids, motors, pneumatic actuators and the like. Accordingly, the invention is not limited by the embodiments described above, but is instead defined by the following claims.

1. A device for removing a needle cannula from an attachment structure, comprising:
a body defining a passageway allowing passage of a needle cannula therethrough while an attachment structure associated with said cannula is restrained in a first position relative to said body;
first and second engagement members, at least one of said engagement members being movable, in an engagement stroke, to cause engagement between said engagement members of a said needle cannula extending along said passageway, at least one of said engagement members being movable, in an extraction stroke following said engaging stroke, away from said first position while firmly gripping a said needle cannula engaged between said engagement members, said movement during the extraction stroke being such as to maintain a generally constant spacing between said engagement members during said extraction stroke, said generally constant spacing varying in relation to a thickness of the engaged needle cannula, such that engaged needle cannula of different thicknesses may be firmly gripped throughout a said extraction stroke.

2. A device according to claim 1, wherein during said engagement stroke the first engagement member is pivotable about a first pivot axis toward the second engagement member.

3. A device according to claim 2, wherein said pivot axis is mounted for translational movement toward and away from the second engagement member.

4. A device according to claim 2, wherein during said extraction stroke both of said first and second engagement members, and a said needle cannula engaged therebetween, pivot together as a unit about a second pivot axis spaced from said first pivot axis.

5. A device according to claim 4, wherein said first and second engagement members are mounted, respectively, on first and second linkages that are pivotally attached to said body, said first pivot axis being established by a pivotal attachment of said first linkage to said second linkage, said second pivot axis being established by a pivotal attachment of said second linkage to said body.

6. A device according to claim 5, further comprising an operation handle pivotally mounted relative to said body and being configured to effect pivotal movement of the first linkage, and the first engagement member mounted thereon, in said engagement stroke.

7. A device according to claim 6, wherein said operation handle is engageable with said first linkage in such a manner as to provide an increased extraction force by way of mechanical leverage that increases as the handle is moved through said engagement stroke.

8. A device according to claim 7, wherein said operation handle is engageable with said first linkage in such a manner that said extraction force decreases, and angular displacement of said first linkage increases, with a continued rotation of said operation handle in the extraction stroke.

9. A device according to claim 7, wherein said operation handle is pivotably mounted to said body for pivotal movement into and out of operable engagement with said first linkage.

10. A device according to claim 6, further comprising an elongated hand grip structure attached to said body, said operation handle being pivotable toward said handgrip structure during said engagement stroke and said extraction stroke.

11. A device according to claim 10, wherein said handgrip structure provides, in general alignment with said passage, a cavity for receiving a needle cannula extracted from its attachment structure in a said extraction stroke.

12. A device according to claim 11, wherein said handgrip structure and cavity thereof provide a containment vessel for containing extracted needle cannulas.

13. A device according to claim 5, further comprising a biasing member biasing said second linkage to place the second engagement member mounted thereon in an initial needle cannula engagement position, said biasing member providing a resistive force opposing pivotal movement of said second linkage during said extraction stroke.

14. A device according to claim 5, further comprising a biasing member biasing said first linkage to a rest position spacing the first engagement member away from said second engagement member.

15. A device according to claim 2, wherein during said extraction stroke said first engagement member continues its pivotal movement toward the second engagement member, and wherein at least one of said engagement members is mounted for translational movement away from the other, said translational movement occurring in relation to said pivotal movement of the first engagement member against a said needle cannula engaged between the first and second engagement members, in such a manner as to maintain said generally constant spacing.

16. A device according to claim 15, wherein said first engagement member is mounted for said translational movement away from said engagement member, in addition to said pivotal movement.

17. A device according to claim 16, further comprising a biasing member for biasing said first engagement member for translation toward said second engagement member, wherein said translational movement away from said second engagement member occurs against a bias of said biasing member.

18. A device according to claim 17, further comprising an adjustment mechanism permitting adjustment of a bias force of said biasing member.

19. A device according to claim 18, wherein said biasing member comprises a compression spring retained within a passage formed in said body, and said adjustment mechanism comprises a stopper threadably received within said passage.

20. A device according to claim 15, wherein said second engagement member is mounted for said translational movement.

21. A device according to claim 15, wherein said second engagement member comprises a backing member along which a needle cannula extending along said passageway is moved as said needle cannula is being gripped and pulled away from said first position by the first engagement member during said extraction stroke.

22. A device according to claim 21, wherein said backing member is configured to slidably engage a needle cannula extending along said passageway, as said needle cannula is being gripped and pulled away from said first position by the first engagement member during said extraction stroke.

23. A device according to claim 21, wherein said backing member is mounted for translational movement toward and away from said first engagement member.

24. A device according to claim 1, wherein said second engagement member is a backing member configured to movably engage a needle cannula extending along said passageway as said needle cannula is being gripped and pulled away from said opening by the first engagement member during said extraction stroke.

25. A device according to claim 1, said body further defining an opening in general alignment with said passageway, for permitting a said needle cannula to pass therethrough and into said passageway, wherein a body portion adjacent said opening serves to establish said first position at a side of said opening opposite said passageway.

26. A device according to claim 25, further comprising an internally tapered guide structure surrounding said opening, for guiding insertion of a said needle cannula into said opening.

27. A device according to claim 26, said guide structure further comprising an internal shoulder portion for positioning and maintaining in a generally upright position a syringe associated with a needle cannula inserted into said opening.

28. A device according to claim 2, wherein said first engagement member is attached to a lever that is pivotally mounted to said body, wherein an extension of said lever provides a hand graspable operation handle.

29. A device according to claim 28, further comprising an elongated hand grip structure extending from said body, said operation handle being pivotable toward said hand grip structure during said engagement stroke and said gripping stroke.

30. A device according to claim 28, wherein said first engagement member is positioned distally of the second engagement member and comprises a gripping surface facing proximally, toward a distally facing surface of said second engagement member.

31. A device according to claim 1, wherein during said extraction stroke said first engagement member is movable toward the second engagement member and the second engagement member is mounted for movement away from the first engagement member in relation to movement of the first engagement member toward said second engagement member and against a said needle cannula engaged between said first and second engagement members, said movement of the second engagement member being in such a manner as to maintain said generally constant spacing during said extraction stroke.

32. A device according to claim 31, further comprising a biasing member for biasing said second engagement member toward said first engagement member, wherein said movement away from said first engagement member occurs against a bias of said biasing member.

33. A device according to claim 32, further comprising an adjustment mechanism permitting adjustment of a bias force of said biasing member.

34. A device according to claim 33, wherein said biasing member comprises a compression spring retained within a passage in said body, and said adjustment mechanism comprises a stopper threadably received within said passage.

35. A device according to claim 31, wherein said second engagement member is a backing member along which a needle cannula extending along said passageway is, during said extraction stroke, moved as said needle cannula is being gripped and pulled away from said opening by the first engagement member.

36. A device according to claim 35, wherein said backing member is configured to rotatably engage a needle cannula extending along said passageway, as said needle cannula is being gripped and pulled away from said first position by the first engagement member during said extraction stroke.
